(19) United States
(12) Patent Application Publication
(10) Pub. No.: US 2011/0001042 A1
(43) Pub. Date: Jan. 6, 2011

(54) ASSESSING TREATMENT COMPLIANCE

(76) Inventors: John B. Hagan, Rochester, MN (US); Robert L. Taylor, Rochester, MN (US); Ravinder J. Singh, Rochester, MN (US); Jesse C. Seegmiller, Rochester, MN (US); Brian C. Netzel, Rochester, MN (US); Nichole L. Korpi-Steiner, Rochester, MN (US)

Correspondence Address: FISH & RICHARDSON P.C. (TC), PO BOX 1022, MINNEAPOLIS, MN, 55440-1022, US

(21) Appl. No.: 12/808,109

(22) PCT Filed: Dec. 12, 2008
(86) PCT No.: PCT/US08/86678

§ 371 Date: Sep. 9, 2010

Related U.S. Application Data
(60) Provisional application No. 61/013,881, filed on Dec. 14, 2007.

(51) Int. Cl. G01N 33/493; H01J 49/26

(52) U.S. Cl. 250/282; 73/53.01

(57) ABSTRACT

This document provides methods and materials related to determining whether or not a mammal is receiving a steroid treatment. For example, methods and materials involved in assessing a biological sample (e.g., a urine sample) from a mammal for the presence or absence of a steroid or a steroid metabolite to determine whether or not the mammal is complying with a steroid treatment (e.g., a corticosteroid treatment for an allergic disease or asthma) are provided.

### CROSS-REFERENCE TO RELATED APPLICATIONS

This application claims the benefit of U.S. Provisional Application Ser. No. 61/013,881, filed Dec. 14, 2007.

### BACKGROUND

1. Technical Field

This document relates to methods and materials involved in determining whether or not a mammal is receiving a steroid treatment. For example, this document relates to methods and materials involved in assessing a biological sample (e.g., a urine sample) from a mammal for the presence or absence of a steroid metabolite to determine whether or not the mammal is complying with a steroid treatment (e.g., a corticosteroid treatment for an allergic disease or asthma).

2. Background Information

Asthma is primarily a chronic inflammatory disease of the airways. This inflammation can cause symptoms such as (a) overly reactive bronchi that are more sensitive to various asthma triggers such as allergens, cold and dry air, smoke and viruses, and (b) airflow obstruction (e.g., difficulty moving air in and out of the lungs). These symptoms are typically manifested by coughing, wheezing, shortness of breath or rapid breathing, and chest tightness.

Various treatments for asthma exist. For example, steroids can be used to treat asthma patients. Unfortunately, asthma is a common disease where patient noncompliance has been associated with excess morbidity, mortality, and costs.

### SUMMARY

This document provides methods and materials related to determining whether or not a mammal is receiving a steroid treatment. For example, this document provides methods and materials involved in assessing a biological sample (e.g., a urine sample) from a mammal for the presence or absence of a steroid metabolite to determine whether or not the mammal is complying with a steroid treatment (e.g., a corticosteroid treatment for an allergic disease or asthma). As described herein, a urine sample can be assessed for the presence or absence of a steroid metabolite (e.g., fluticasone 17β carboxylic acid) to determine whether or not a mammal is complying with a steroid treatment (e.g., inhaled fluticasone propionate for treating an allergic disease or asthma). An inhaled steroid can be inhaled through the mouth or nose into the lungs. Having the ability to identify treatment non-compliance and to monitor proper steroid treatment can allow clinicians to address issues of compliance with the patient or family of the patient, thereby resulting in improved care.

In general, this document features a method for assessing steroid treatment compliance of a mammal (e.g., a human) instructed to administer (e.g., self administer) a steroid. The method comprises determining whether or not a biological sample from the mammal contains a detectable level of the steroid or a metabolite of the steroid, wherein the presence of the detectable level indicates that the mammal is in compliance with the instructed steroid treatment, and wherein the absence of the detectable level indicates that the mammal is not in compliance with the instructed steroid treatment. The steroid can be fluticasone propionate. The metabolite can be fluticasone 17β carboxylic acid. The mammal can have asthma. The mammal can have an allergic disease. The biological sample can be a urine sample.

In another aspect, this document features a method for assessing the metabolic activity of CYP3A4 in a human who received a steroid (e.g., fluticasone propionate). The method comprises, or consists essentially of, (a) using mass spectrometry to determine the level of the steroid (e.g., fluticasone propionate) and the level of a metabolite of the steroid (e.g., fluticasone propionate) present in a biological sample from the human and (b) diagnosing the human as having poor CYP3A4 metabolic activity if the ratio of the steroid (e.g., fluticasone propionate) to the metabolite is greater than that compared to normal humans and diagnosing the human as having active CYP3A4 metabolic activity if the ratio of the steroid (e.g., fluticasone propionate) to the metabolite is less than that compared to normal humans. The method can comprise communicating the diagnosis to the human. The method can comprise inserting a notation of the diagnosis into a medical record for the human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a graph plotting intensity (counts per minute) versus retention time (minutes) for fluticasone propionate and 17β carboxylic acid fluticasone propionate. Both compounds were analyzed using various ion pairs with the use of multiple reaction monitoring (MRM). The liquid chromatography employed utilized isocratic and gradient elution conditions on a reversed phase column.

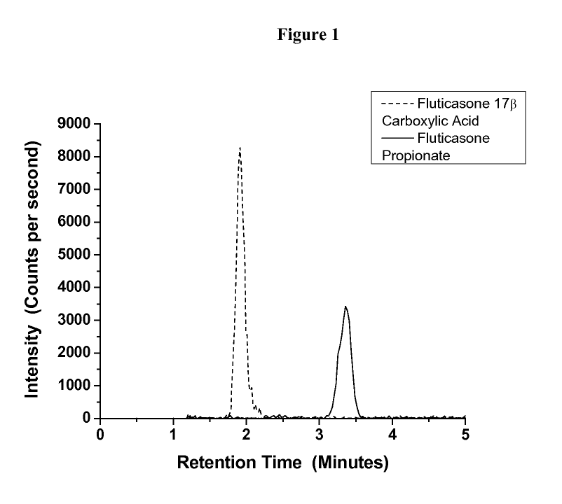

FIG. 2 contains two mass spectrometry chromatograms plotting intensity (counts per minute) versus retention time (minutes) for actual patient urine samples. FIG. 2A is from a patient not using fluticasone propionate, and FIG. 2B is from a patient who is taking fluticasone propionate. FIG. 2B demonstrates that the detection of actual levels of 17β carboxylic acid fluticasone propionate is observed in the urine.

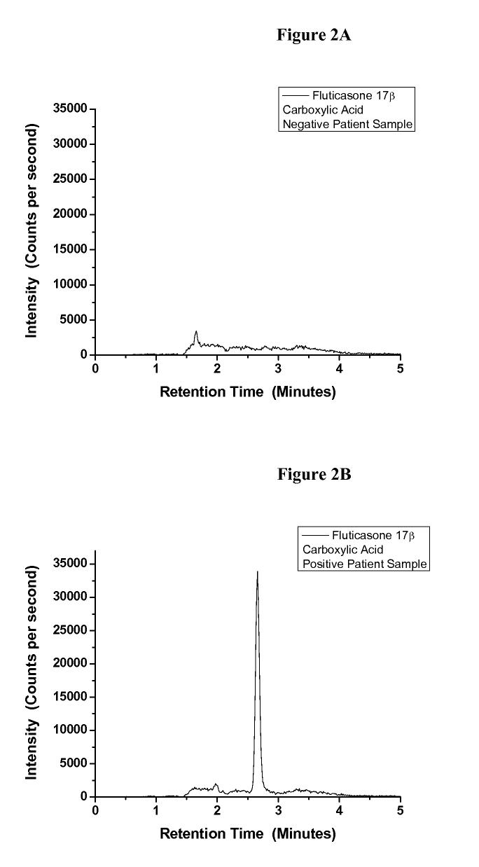

### DETAILED DESCRIPTION

This document provides methods and materials related to determining whether or not a mammal is receiving a steroid treatment. For example, this document provides methods and materials involved in assessing a biological sample (e.g., a urine sample) from a mammal for the presence or absence of a steroid or a steroid metabolite to determine whether or not the mammal is complying with a steroid treatment (e.g., a corticosteroid treatment for an allergic disease or asthma). As described herein, if a biological sample from a mammal contains a detectable level of a steroid or steroid metabolite, then the mammal can be classified as complying with a steroid treatment. If a biological sample from a mammal does not contain a detectable level of a steroid or steroid metabolite, then the mammal can be classified as not complying with a steroid treatment.

A steroid treatment can be any type of steroid treatment (e.g., chronic or acute treatment) designed to treat any type of disease or condition including, without limitation, asthma, rheumatologic conditions such as rheumatoid arthritis and lupus erythematosis, musculoskeletal processes such as those causing pain, gastrointestinal diseases such as inflammatory bowel diseases, allergic diseases such as allergic rhinitis, chronic rhinosinusitis, angioedema, and anaphylaxis, and various dermatologic conditions. Most steroids such as fluticasone propionate can undergo metabolism through cytochrome P450 3A4 (CYP3A4). Measurement of a steroid and/or its corresponding metabolite(s) including, without limitation, fluticasone propionate and the fluticasone propionate 17β carboxylic acid metabolite, can provide a diagnostic means to evaluate the metabolic function of CYP3A4. In some cases, the metabolic function of CYP3A4 (and possible pharmacogenomic variation responsible for the function) can explain the heterogeneity of clinical effects as well as adverse effects (including but not limited to conditions such as steroid psychosis, osteoporosis, an altered growth) of steroids and other pharmacologic agents that can undergo metabolism by CYP3A4. Examples of steroids include, without limitation, prednisone, triamcinolone acetonide, mometasone furoate, budesonide, fluticasone furoate, flunisolide, fluticasone propionate, and other corticosteroids such as beclomethasone, dexamethasone, methylprednisolone, and prednisolone.

A steroid metabolite can be any metabolite of a steroid that is capable of indicating that a particular steroid or a steroid of a particular class of steroids was administered to a mammal. Examples of steroid metabolites include, without limitation, fluticasone propionate 17β carboxylic acid, 9,11-epoxy mometasone furoate, fluticasone furoate 17B carboxylic acid, the 6-beta-hydroxy metabolites of flunisolide, budesonide, and triamcinolone acetonide.

Any type of mammal can be assessed using the methods and materials provided herein to determine whether or not the mammal is receiving a steroid treatment. For example, the methods and materials provided herein can be used to assess a human, dog, cat, horse, cow, goat, sheep, rat, or mouse. In some cases, the mammal can be a human that has asthma and was instructed to self administer a steroid treatment.

Any appropriate biological sample can be evaluated to determine if it contains one or more steroids or steroid metabolites. For example, blood (e.g., peripheral blood or venous prostate blood), plasma, serum, sputum, saliva, urine, semen, and/or seminal fluid can be evaluated to determine if the sample contains one or more steroids or steroid metabolites. Any appropriate method can be used to obtain a biological sample from a mammal. For example, a blood sample can be obtained by peripheral venipuncture, and urine samples can be obtained using standard urine collection techniques. A sample can be manipulated prior to being evaluated for the level of one or more steroids or steroid metabolites.

Any appropriate method can be used to evaluate a biological sample for a detectable level of a steroid or a steroid metabolite. For example, mass spectrometry can be used to determine whether or not a biological sample (e.g., a urine sample) contains a detectable level of a steroid (e.g., FP) or a steroid metabolite (e.g., fluticasone 17β carboxylic acid). In some cases, a mass spectrometry analysis can be performed by any appropriate method that can resolve a steroid or a steroid metabolite and a corresponding isotopically-labeled internal standard and/or an external standard. Instruments that can be used for this assay include, without limitation, API 3000, 4000, or 5000 (Applied Biosystems) and/or other comparable tandem mass spectrometers from various vendors. For example, when the steroid metabolite is fluticasone 17β carboxylic acid, various selective MRM transitions can be used. Complete analysis time under these conditions can be about 3.5 minutes or less with fluticasone 17β carboxylic acid eluting after about two minutes. Other mass spectrometry methods also can be used for sample analysis, e.g., gas chromatography mass spectrometry (GC-MS/MS).

In some cases, a liquid chromatography tandem mass spectrometry (LC-MS/MS) profile can be generated by the isotopically labeled internal standard that allows for the rapid identification of the unlabeled steroid or steroid metabolite in the same sample. Levels of a steroid or steroid metabolite can be calculated in conjunction with a standard calibration curve that can be run in parallel with the samples of interest. A standard calibration curve is typically generated by LC-MS/MS analysis of increasing concentrations, within an empirically determined measurable range, of the reaction product in the presence of a constant amount of the isotopically labeled internal standard. Any appropriate data processing software can be used to analyze the results.

The invention will be further described in the following examples, which do not limit the scope of the invention described in the claims.

### EXAMPLES

### Example 1

### Detecting Fluticasone Propionate and a Metabolite of Fluticasone Propionate

Various MRM transitions were monitored which included 501.2-313.2, 501.2-293.3 ion pairs from fluticasone propionate (FP) and were used to develop a sensitive assay for detecting FP. A deuterium labeled FP (d5-FP) was also spiked into all standards, samples, and controls and was used as an internal standard. Briefly, samples were extracted using a liquid liquid extraction technique. First, the internal standard was spiked into each sample. Then, sample protein was removed using acetonitrile. Methylene chloride was added to partition the steroids into the organic phase. The aqueous layer was then aspirated, and the extracted was washed using a base wash and aspirated, followed by an acid wash and aspirated and finally washed with water and aspirated. The organic extract was then dried in a nitrogen chamber and reconstituted. However, a solid phase extraction for this would also be suitable.

Using PBS/1% BSA, the intra-assay variability observed was 0.82, 8.36, 2.74, 1.79, 1.24, 2.61, 1.58, 0.57, and 2.73 for the concentration of 5, 10, 20, 40, 80, 160, 320, 640, and 1280 pg/mL of FP, respectively, were determined by mass spectrometry, and is listed in Table 1. Briefly, a sample preparation method was used to extract the steroids of interest from real and synthetic sample matrices. The samples were then analyzed using an LC-MS/MS system using isocratic and gradient elution LC techniques. These results indicate that low levels of FP can be detected in real and synthetic matrices in a reliable fashion with % CVs less than 10% across a wide concentration range.

TABLE 1
Results without 1280 calibrator.

| | 5EX | 10EX | 20EX | 40EX | 80EX | 160EX | 320EX | 640EX |
|---|---|---|---|---|---|---|---|---|
| RUN1 | 6.89 | 10.4 | 18.1 | 38.3 | 80.2 | 165 | 314 | 642 |
| RUN2 | 6.56 | 10.5 | 20.9 | 39.2 | 81.3 | 157 | 321 | 659 |
| RUN3 | 6.42 | 10.8 | 20.1 | 41.2 | 81.5 | 162 | 306 | 646 |
| RUN4 | 6.45 | 11 | 21.2 | 37.7 | 79.9 | 167 | 311 | 651 |
| RUN5 | 6.99 | 10.7 | 22.3 | 40.1 | 78.2 | 165 | 316 | 646 |
| RUN6 | 6.56 | 11.2 | 21.2 | 39.9 | 74.9 | 162 | 321 | 677 |
| RUN7 | 8.49 | 12.8 | 21.1 | 38.8 | 79.6 | 160 | 314 | 642 |
| RUN8 | 8.6 | 12.4 | 21.5 | 40 | 73.9 | 162 | 313 | 644 |
| RUN9 | 8.71 | 13.1 | 27.5 | 42.5 | 82.1 | 163 | 339 | 662 |
| RUN10 | 9.06 | 15.4 | 25.2 | 44.3 | 82.8 | 166 | 308 | 667 |
| RUN11 | 4.42 | 8.12 | 18 | 40.8 | 85.6 | 156 | 323 | 639 |
| RUN12 | 2.07 | 7.64 | 17.4 | 38.7 | 80.2 | 167 | 318 | 687 |
| RUN13 | 3.16 | 6.45 | 16.6 | 37.1 | 76.4 | 173 | 309 | 649 |
| RUN14 | 2.31 | 9.18 | 17.3 | 36.9 | 80 | 160 | 335 | 678 |
| RUN15 | 4.84 | 11.4 | 19.9 | 39.5 | 76.9 | 150 | 315 | 657 |
| RUN16 | 4.8 | 11.6 | 18.9 | 40.4 | 83 | 157 | 321 | 658 |
| RUN17 | 4.88 | 9.89 | 19.1 | 39 | 79.7 | 158 | 325 | 651 |
| Average | 5.95 | 10.74 | 20.37 | 39.67 | 79.78 | 161.76 | 318.18 | 656.18 |
| StdDev | 2.180487 | 2.149881 | 2.835658 | 1.877087 | 3.011131 | 5.356415 | 8.924932 | 14.09892 |
| % CV | 36.62512 | 20.01751 | 13.92035 | 4.731684 | 3.77446 | 3.311238 | 2.805026 | 2.148647 |
| Accuracy | 119.0706 | 107.4 | 101.8529 | 99.17647 | 99.72059 | 101.1029 | 99.43015 | 102.5276 |

Stripped serum samples spiked with FP served as a real sample matrix and were evaluated with standard curves using 0, 5, 10, 20, 40, 80, 160, 320, 640, and 1280 pg/mL. Using these curves, results were obtained demonstrating that the sensitivity of the mass spectrometry to measure FP was on the order of about 10 pg/mL (Table 1). Visualization of the FP peak was performed consistently at the 5 pg/mL concentration (Table 2). There was no significant difference observed with or without the inclusion of the 1280 pg/mL data point in the capacity of mass spectrometry to evaluate the samples at the 10 pg/mL dilution (Table 2). Table 2 contains a series of tables demonstrating the assay accuracy. The 1% bovine serum albumin (BSA) standards were run in triplicate generating the % coefficient of variation (% CV) data. The values for the fluticasone propionate are denoted and represent the pg/mL concentration value. The stripped serum standards (SS) are also reported in pg/mL, and the assay % CV along with the accuracy is listed.

TABLE 2
PBS 1% BSA Standards.

| Summary 5 pg/ml | N = 3 | Accuracy | Summary 160 pg/ml | N = 3 | Accuracy |
|---|---|---|---|---|---|
| Ave | 4.89 | 97.80% | Ave | 154.6667 | 96.67% |
| Std | 0.04 | | Std | 4.041452 | |
| % CV | 0.817996 | | % CV | 2.613008 | |
| Summary 10 pg/ml | N = 3 | Accuracy | Summary 320 pg/ml | N = 3 | Accuracy |
| Ave | 10.97333 | 109.73% | Ave | 319.3333 | 99.79% |
| Std | 0.917678 | | Std | 5.033223 | |
| % CV | 8.362803 | | % CV | 1.576166 | |
| Summary 20 pg/ml | N = 3 | Accuracy | Summary 640 pg/ml | N = 3 | Accuracy |
| Ave | 19.3 | 96.50% | Ave | 653.3333 | 102.08% |
| Std | 0.52915 | | Std | 3.785939 | |
| % CV | 2.741711 | | % CV | 0.57948 | |
| Summary 40 pg/ml | N = 3 | Accuracy | Summary 160 pg/ml | N = 3 | Accuracy |
| Ave | 39.53333 | 98.83% | Ave | 1320 | 103.13% |
| Std | 0.70946 | | Std | 36.05551 | |
| % CV | 1.794587 | | % CV | 2.731478 | |
| Summary 80 pg/ml | N = 3 | Accuracy | | | |
| Ave | 79.6333 | 99.54% | | | |
| Std | 0.989949 | | | | |
| % CV | 1.243135 | | | | |
| Summary 5SS* | N = 10 | Accuracy | | | |
| Ave | 8.62 | 172.40% | | | |
| Std | 2.216138 | | | | |
| % CV | 25.70926 | | | | |
| Summary 10SS | N = 5 | Accuracy | | | |
| Ave | 11.24 | 112.40% | | | |
| Std | 1.21161 | | | | |
| % CV | 10.77945 | | | | |
| Summary 20SS | N = 5 | Accuracy | | | |
| Ave | 19.58 | 97.90% | | | |
| Std | 2.03519 | | | | |
| % CV | 10.39423 | | | | |

*SS Denotes Stripped Serum Standard Matrix in pg/mL

TABLE 3
Results using 1280 pg/mL calibrator.

| | 5EX | 10EX | 20EX | 40EX | 80EX | 160EX | 320EX | 640EX | 1280EX |
|---|---|---|---|---|---|---|---|---|---|
| RUN1 | 4.89 | 11.40 | 19.90 | 39.40 | 76.70 | 150.00 | 314.00 | 655.00 | 1280.00 |
| RUN2 | 4.85 | 11.60 | 18.90 | 40.30 | 82.70 | 157.00 | 320.00 | 656.00 | 1350.00 |
| RUN3 | 4.93 | 9.92 | 19.10 | 38.90 | 79.50 | 157.00 | 324.00 | 649.00 | 1330.00 |
| RUN4 | 5.60 | 9.61 | 19.30 | 41.90 | 86.10 | 156.00 | 321.00 | 632.00 | 1280.00 |
| RUN5 | 3.64 | 9.14 | 18.70 | 39.80 | 80.80 | 167.00 | 315.00 | 680.00 | 1390.00 |
| RUN6 | 4.71 | 7.96 | 18.00 | 38.20 | 77.00 | 172.00 | 307.00 | 642.00 | 1350.00 |
| RUN7 | 3.88 | 10.70 | 18.70 | 38.00 | 80.60 | 160.00 | 333.00 | 671.00 | 1360.00 |
| RUN8 | 6.15 | 11.10 | 20.30 | 42.30 | 80.20 | 173.00 | 327.00 | 597.00 | 1300.00 |
| RUN9 | 7.38 | 12.10 | 21.10 | 44.30 | 86.20 | 165.00 | 338.00 | 730.00 | 1390.00 |
| RUN10 | 7.36 | 13.90 | 24.30 | 43.50 | 88.10 | 174.00 | 322.00 | 701.00 | 1400.00 |
| RUN11 | 7.47 | 12.00 | 22.80 | 40.30 | 80.30 | 159.00 | 375.00 | 633.00 | 1350.00 |
| RUN12 | 10.50 | 14.70 | 23.20 | 41.50 | 74.70 | 161.00 | 310.00 | 635.00 | 1300.00 |
| RUN13 | 10.60 | 14.30 | 29.20 | 43.90 | 82.80 | 162.00 | 309.00 | 652.00 | 1290.00 |
| RUN14 | 10.70 | 15.00 | 26.90 | 45.60 | 83.50 | 165.00 | 335.00 | 658.00 | 1310.00 |
| RUN15 | 11.00 | 17.20 | 23.00 | 43.50 | 82.60 | 160.00 | 305.00 | 631.00 | 1290.00 |
| RUN16 | 9.73 | 14.00 | 24.10 | 40.10 | 81.10 | 165.00 | 301.00 | 636.00 | 1290.00 |
| RUN17 | 9.77 | 14.20 | 25.20 | 42.50 | 79.40 | 164.00 | 306.00 | 631.00 | 1320.00 |
| RUN18 | 10.30 | 13.90 | 24.10 | 42.20 | 76.20 | 161.00 | 311.00 | 661.00 | 1330.00 |
| RUN19 | 9.87 | 14.40 | 25.30 | 44.00 | 83.00 | 162.00 | 315.00 | 606.00 | 1270.00 |
| RUN20 | 14.80 | 18.00 | 27.90 | 44.80 | 84.00 | 155.00 | 301.00 | 622.00 | 1300.00 |
| RUN21 | 14.50 | 18.20 | | | | | 307.00 | 689.00 | 1330.00 |
| Average | 8.22 | 13.02 | 22.50 | 41.75 | 81.28 | 162.25 | 318.86 | 650.81 | 1324.29 |
| StdDev | 3.30 | 2.84 | 3.37 | 2.28 | 3.50 | 6.15 | 16.81 | 31.18 | 38.80 |
| % CV | 40.14 | 21.78 | 14.96 | 5.46 | 4.31 | 3.79 | 5.27 | 4.79 | 2.93 |
| Accuracy | 164.4095 | 130.1571 | 112.5 | 104.375 | 101.5938 | 101.4063 | 99.64286 | 101.689 | 103.4598 |

A mass spectrometry assay was developed to detect a major metabolite of FP, fluticasone 17β carboxylic acid. Briefly, a modification to the extraction method noted above with the use of an acid extraction method coupled with LC-MS/MS detection was used. Fluticasone 17β carboxylic acid was obtained and used as a source material. Again various MRM's were monitored including 453.2-293.1 and 453.2-275.2 transitions, and it was observed that this method could detect circulating levels of the metabolite present in a real patient sample. These results suggest that the simultaneous analysis of FP and fluticasone 17β carboxylic acid can be used to monitor compliance and metabolic efficiency.

These results demonstrate that FP and metabolites of FP can be detected using mass spectrometry. In addition, these results demonstrate that the methods and materials provided herein can be used to detect FP and fluticasone 17β carboxylic acid in the same sample.

### Example 2

### Detecting Fluticasone-17Ω Carboxylic Acid in Urine by LC-MS/MS

Fluticasone-17β carboxylic acid was extracted from urine using an acetonitrile precipitation followed by methylene chloride liquid extraction of the supernatant. Sixty μL of the reconstituted sample extract was analyzed by LC-MS/MS (ABI 4000). The linearity, precision, recovery and limit of quantitation (LOQ) were determined. Measurement of fluticasone-17β carboxylic acid in urine collected daily from patients before (days 1-2), during (days 3-6; total dose Flovent 110 2 puffs daily), and following cessation of FP therapy (days 7-14) was conducted (n=4).

The linear range of fluticasone-17β carboxylic acid measured by LC-MS/MS was 10-9510 pg/mL. The LOQ with a CV<20% was 10 pg/mL, and recovery ranged from 85.8-111.9% with a mean of 97.9%. Within-run precision testing indicated a maximum CV of 9.3% at 10.3 pg/mL. Between-run precision CVs for urine pools spiked with 3 concentrations of fluticasone-17 β carboxylic acid were 10.6% at 11.1 pg/mL, 11.2% at 500.9 pg/mL, and 8.7% at 5116 pg/mL. Detection of fluticasone-17β carboxylic acid in urine from patients prior to FP therapy was <10 pg/mL (days 1 & 2), ranged from 157-1830 pg/mL when receiving therapy (days 3-6) and was undetectable 5 days after stopping therapy (days 11-14).

Measurement of fluticasone-17β carboxylic acid by LC-MS/MS exhibited acceptable analytical performance for clinical use. These results support the clinical utility of measuring fluticasone-17β carboxylic acid in urine to monitor patient compliance with FP therapy.

### Example 3

### Analysis of Synthetic Corticosteroids in Biologic Secretions

In order to evaluate the sensitivity and specificity of fluticasone 17β carboxylic acid, nineteen patients with asthma were recruited. Nine of these subjects were on treatment with fluticasone propionate. As a gold standard of compliance, treatment was documented by an RN study nurse who witnessed the patients' administration of fluticasone propionate 16-24 hours prior to submission of a urine sample for the analysis of fluticasone 17 β carboxylic acid. Urine samples from ten subjects with asthma but not receiving fluticasone propionate were used as controls.

The results revealed that the sensitivity and specificity of mass spectrometry to detect 17 β fluticasone propionate within 16-24 hours of administration was 100% and 100%, respectively (Table 4).

TABLE 4

| | Fluticasone | | |
|---|---|---|---|
| | Administered | Fluticasone NOT | |
| | Under Observation | Administered | Total |
| Positive Urine for | 9 | 0 | 9 |
| fluticasone 17β | | | |
| carboxylic acid | | | |
| Negative Urine for | 0 | 10 | 10 |
| fluticasone 17β | | | |
| carboxylic acid | | | |
| Total | 9 | 10 | 19 |

### Example 4

### Analysis of Urine Samples with and without Preservatives

Fluticasone 17β carboxylic acid was measured in urine samples lacking preservatives or urine samples containing one of the following preservatives: boric acid, glacial acetic acid, HCL, toluene, or sodium bicarbonate (Table 5).

TABLE 5

| | | URINE-BORIC | | URINE-ACETIC | | | | | | | |
|---|---|---|---|---|---|---|---|---|---|---|---|
| URINE-NO | | ACID | | ACID | | URINE-HCL | | URINE-Na2CO3 | | URINE-Toluene | |
| PRESERV | | | % Difference | Glacial | % Difference | | % Difference | | % Difference | | % Difference |
| Spec. | No Preser- | Boric | between No | Acetic | between No | | between No | Sodium | between No | | between No |
| ID | vatives | Acid | Pres & BA | Acid | Pres & GAA | HCL | Pres & HCL | Bicarbonate | Pres & Na2CO3 | Toluene | Pres & Toluene |
| 1 | 173 | 172 | −0.6% | 170 | −1.7% | 152 | −12.1% | 9.21 | −94.7% | 190 | 9.8% |
| 2 | 318 | 317 | −0.3% | 258 | −18.9% | 272 | −14.5% | 23.7 | −92.5% | 323 | 1.6% |
| 3 | 461 | 484 | 5.0% | 540 | 17.1% | 503 | 9.1% | 26.7 | −94.2% | 412 | −10.6% |

| | | | | | |
|---|---|---|---|---|---|
| Mean | 1.4% | −1.2% | −5.8% | −93.8% | 0.3% |

These results indicate that acceptable specimens include urine without preservatives or urine containing the following preservatives: boric acid, glacial acetic acid, HCL, or toluene. Sodium bicarbonate preservative did not appear acceptable.

Fluticasone 17β carboxylic acid was measured in pooled urine samples lacking preservatives that were stored at ambient temperature, were refrigerated, or were frozen and subjected to freeze/thaw cycles (Table 6).

TABLE 6
Ambient

| Spec. ID | Day 0 | Day 1 | % Diff. 1 | Day 3 | % Diff. 3 | Day 7 | % Diff 7 |
|---|---|---|---|---|---|---|---|
| no pres | 173 | 172 | −0.6% | 170 | −1.7% | 176 | 1.7% |
| no pres | 318 | 309 | −2.8% | 306 | −3.8% | 332 | 4.4% |
| no pres | 461 | 393 | −14.8% | 497 | 7.8% | 616 | 33.6% |
| Mean | | | −6.1% | | 0.8% | | 13.3% |

Refrigerated

| Spec. ID | Day 0 | Day 1 | % Diff. 1 | Day 3 | % Diff. 3 | Day 7 | % Diff 7 |
|---|---|---|---|---|---|---|---|
| no pres | 173 | 160 | −7.5% | 186 | 7.5% | 143 | −17.3% |
| no pres | 318 | 304 | −4.4% | 267 | −16.0% | 309 | −2.8% |
| no pres | 461 | 408 | −11.5% | 434 | −5.9% | 415 | −10.0% |
| Mean | | | −7.8% | | −4.8% | | −10.0% |

Freeze/Thaw

| Spec. ID | F/T 0 | F/T 1 | % Diff. 1 | F/T 2 | % Diff. 2 | F/T 3 | % Diff 3 |
|---|---|---|---|---|---|---|---|
| no pres | 173 | 173 | 0.0% | 177 | 2.3% | 183 | 5.8% |
| no pres | 318 | 297 | −6.6% | 307 | −3.5% | 280 | −11.9% |
| no pres | 461 | 433 | −6.1% | 420 | −8.9% | 445 | −3.5% |
| Mean | | | −4.2% | | −3.3% | | −3.2% |

Fluticasone 17β carboxylic acid was stable in non-preserved urine stored at ambient temperature for 3 days, refrigerated for 7 days, or frozen with up to 3 freeze-thaw cycles.

Fluticasone 17β carboxylic acid was measured in pooled urine samples containing boric acid as a preservative that were stored at ambient temperature, were refrigerated, or were frozen and subjected to freeze/thaw cycles (Table 7).

TABLE 7
Ambient

| Spec. ID | Day 0 | Day 1 | % Diff. 1 | Day 3 | % Diff. 3 | Day 7 | % Diff 7 |
|---|---|---|---|---|---|---|---|
| Boric Acid | 172 | 170 | −1.2% | 174 | 1.2% | 175 | 1.7% |
| Boric Acid | 317 | 288 | −9.1% | 313 | −1.3% | 283 | −10.7% |
| Boric Acid | 484 | 392 | −19.0% | 446 | −7.9% | 429 | −11.4% |
| Mean | | | −9.8% | | −2.7% | | −6.8% |

Refrigerated

| Spec. ID | Day 0 | Day 1 | % Diff. 1 | Day 3 | % Diff. 3 | Day 7 | % Diff 7 |
|---|---|---|---|---|---|---|---|
| Boric Acid | 172 | 164 | −4.7% | 171 | −0.6% | 147 | −14.5% |
| Boric Acid | 317 | 297 | −6.3% | 294 | −7.3% | 285 | −10.1% |
| Boric Acid | 484 | 399 | −17.6% | 416 | −14.0% | 405 | −16.3% |
| Mean | | | −9.5% | | −7.3% | | −13.7% |

Freeze/Thaw

| Spec. ID | F/T 0 | F/T 1 | % Diff. 1 | F/T 2 | % Diff. 2 | F/T 3 | % Diff 3 |
|---|---|---|---|---|---|---|---|
| Boric Acid | 172 | 156 | −9.3% | 157 | −8.7% | 163 | −5.2% |
| Boric Acid | 317 | 300 | −5.4% | 250 | −21.1% | 264 | −16.7% |
| Boric Acid | 484 | 435 | −10.1% | 380 | −21.5% | 386 | −20.2% |
| Mean | | | −8.3% | | −17.1% | | −14.1% |

Fluticasone 17β carboxylic acid was stable in boric acid-preserved urine stored at ambient temperature for 7 days, refrigerated for 3 days, or frozen with up to 1 freeze-thaw cycle.

Fluticasone 17β carboxylic acid was measured in pooled urine samples containing glacial acetic acid as a preservative that were stored at ambient temperature, were refrigerated, or were frozen and subjected to freeze/thaw cycles (Table 8).

TABLE 8
Ambient

| Spec. ID | Day 0 | Day 1 | % Diff. 1 | Day 3 | % Diff. 3 | Day 7 | % Diff 7 |
|---|---|---|---|---|---|---|---|
| Glacial Acetic Acid | 170 | 140 | −17.6% | 159 | −6.5% | 163 | −4.1% |
| Glacial Acetic Acid | 258 | 269 | 4.3% | 296 | 14.7% | 296 | 14.7% |
| Glacial Acetic Acid | 540 | 455 | −15.7% | 502 | −7.0% | 479 | −11.3% |
| Mean | | | −9.7% | | 0.4% | | −0.2% |

Refrigerated

| Spec. ID | Day 0 | Day 1 | % Diff. 1 | Day 3 | % Diff. 3 | Day 7 | % Diff 7 |
|---|---|---|---|---|---|---|---|
| Glacial Acetic Acid | 170 | 146 | −14.1% | 162 | −4.7% | 141 | −17.1% |
| Glacial Acetic Acid | 258 | 255 | −1.2% | 286 | 10.9% | 252 | −2.3% |
| Glacial Acetic Acid | 552 | 444 | −19.6% | 542 | −1.8% | 449 | −18.7% |
| Mean | | | −11.6% | | 1.4% | | −12.7% |

Freeze/Thaw

| Spec. ID | F/T 0 | F/T 1 | % Diff. 1 | F/T 2 | % Diff. 2 | F/T 3 | % Diff 3 |
|---|---|---|---|---|---|---|---|
| Glacial Acetic Acid | 170 | 144 | −15.3% | 166 | −2.4% | 145 | −14.7% |
| Glacial Acetic Acid | 258 | 266 | 3.1% | 253 | −1.9% | 258 | 0.0% |
| Glacial Acetic Acid | 552 | 477 | −13.6% | 480 | −13.0% | 463 | −16.1% |
| Mean | | | −8.6% | | −5.8% | | −10.3% |

Fluticasone 17β carboxylic acid was stable in glacial acetic acid -preserved urine stored at ambient temperature for 7 days or frozen with up to 2 freeze-thaw cycles.

Fluticasone 17β carboxylic acid was measured in pooled urine samples containing hydrochloric acid as a preservative that were stored at ambient temperature, were refrigerated, or were frozen and subjected to freeze/thaw cycles (Table 9).

TABLE 9
Ambient

| Spec. ID | Day 0 | Day 1 | % Diff. 1 | Day 3 | % Diff. 3 | Day 7 | % Diff 7 |
|---|---|---|---|---|---|---|---|
| HCL | 152 | 137 | −9.9% | 152 | 0.0% | 150 | −1.3% |
| HCL | 272 | 290 | 6.6% | 314 | 15.4% | 272 | 0.0% |
| HCL | 503 | 419 | −16.7% | 511 | 1.6% | 451 | −10.3% |
| Mean | | | −6.7% | | 5.7% | | −3.9% |

Refrigerated

| Spec. ID | Day 0 | Day 1 | % Diff. 1 | Day 3 | % Diff. 3 | Day 7 | % Diff 7 |
|---|---|---|---|---|---|---|---|
| HCL | 152 | 145 | −4.6% | 152 | 0.0% | 126 | −17.1% |
| HCL | 272 | 265 | −2.6% | 277 | 1.8% | 249 | −8.5% |
| HCL | 503 | 455 | −9.5% | 556 | 10.5% | 460 | −8.5% |
| Mean | | | −5.6% | | 4.1% | | −11.4% |

Freeze/Thaw

| Spec. ID | F/T 0 | F/T 1 | % Diff. 1 | F/T 2 | % Diff. 2 | F/T 3 | % Diff 3 |
|---|---|---|---|---|---|---|---|
| HCL | 152 | 141 | −7.2% | 141 | −7.2% | 138 | −9.2% |
| HCL | 272 | 258 | −5.1% | 217 | −20.2% | 259 | −4.8% |
| HCL | 503 | 419 | −16.7% | 401 | −20.3% | 397 | −21.1% |

Fluticasone 17β carboxylic acid was stable in hydrochloric acid-preserved urine stored at ambient temperature for 7 days, refrigerated for 3 days, or frozen with up to 1 freeze-thaw cycle.

Fluticasone 17β carboxylic acid was measured in pooled urine samples containing toluene as a preservative that were stored at ambient temperature, were refrigerated, or were frozen and subjected to freeze/thaw cycles (Table 10).

TABLE 10
Ambient

| Spec. ID | Day 0 | Day 1 | % Diff. 1 | Day 3 | % Diff. 3 | Day 7 | % Diff 7 |
|---|---|---|---|---|---|---|---|
| Toluene | 190 | 149 | −21.6% | 159 | −16.3% | 172 | −9.5% |
| Toluene | 323 | 261 | −19.2% | 310 | −4.0% | 326 | 0.9% |
| Toluene | 412 | 400 | −2.9% | 489 | 18.7% | 457 | 10.9% |
| Mean | | | −14.6% | | −0.6% | | 0.8% |

Refrigerated

| Spec. ID | Day 0 | Day 1 | % Diff. 1 | Day 3 | % Diff. 3 | Day 7 | % Diff 7 |
|---|---|---|---|---|---|---|---|
| Toluene | 190 | 153 | −19.5% | 154 | −18.9% | 148 | −22.1% |
| Toluene | 323 | 283 | −12.4% | 323 | 0.0% | 279 | −13.6% |
| Toluene | 412 | 365 | −11.4% | 390 | −5.3% | 371 | −10.0% |
| Mean | | | −14.4% | | −8.1% | | −15.2% |

Freeze/Thaw

| Spec. ID | F/T 0 | F/T 1 | % Diff. 1 | F/T 2 | % Diff. 2 | F/T 3 | % Diff 3 |
|---|---|---|---|---|---|---|---|
| Toluene | 190 | 159 | −16.3% | 155 | −18.4% | 156 | −17.9% |
| Toluene | 323 | 293 | −9.3% | 294 | −9.0% | 310 | −4.0% |
| Toluene | 412 | 450 | 9.2% | 426 | 3.4% | 388 | −5.8% |
| Mean | | | −5.5% | | −8.0% | | −9.2% |

Fluticasone 17β carboxylic acid was stable in toluene-preserved urine when immediately frozen with up to 3 freeze-thaw cycles.

The intra-assay precision of measuring fluticasone 17β carboxylic acid in stripped and random urine samples was assessed. Charcoal stripped human urine was spiked to three levels including a low, medium and high level of fluticasone 17β carboxylic acid. Additionally pooled urine (“random”) was spiked to a medium level and monitored to account for possible matrix effects. Each of the four was measured daily for 20 days, and intra-assay precision was found to be acceptable (Table 11).

TABLE 11

| Replicate # | Level I | Level II | Level III | Random Urine |
|---|---|---|---|---|
| 1 | 10.2 | 111.0 | 521.0 | 187 |
| 2 | 9.8 | 108.0 | 555.0 | 189 |
| 3 | 9.6 | 111.0 | 548.0 | 180 |
| 4 | 10.2 | 99.6 | 588.0 | 178 |
| 5 | 9.2 | 106.0 | 564.0 | 189 |
| 6 | 10.7 | 101.0 | 554.0 | 185 |
| 7 | 9.5 | 111.0 | 572.0 | 186 |
| 8 | 9.5 | 108.0 | 582.0 | 195 |
| 9 | 11.0 | 103.0 | 532.0 | 200 |
| 10 | 8.2 | 108.0 | 547.0 | 178 |
| 11 | 10.0 | 103.0 | 586.0 | 197 |
| 12 | 12.4 | 105.0 | 564.0 | 176 |
| 13 | 11.4 | 104.0 | 525.0 | 183 |
| 14 | 10.7 | 106.0 | 535.0 | 191 |
| 15 | 10.3 | 104.0 | 559.0 | 180 |
| 16 | 10.4 | 99.9 | 572.0 | 174 |
| 17 | 11.8 | 111.0 | 566.0 | 187 |
| 18 | 10.0 | 119.0 | 589.0 | 198 |
| 19 | 11.1 | 100.0 | 553.0 | 171 |
| 20 | 10.9 | 118.0 | 566.0 | 181 |
| Mean | 10.3 | 106.8 | 558.9 | 185.3 |
| SD | 1.0 | 5.5 | 20.2 | 8.2 |
| % CV | 9.3% | 5.2% | 3.6% | 4.4% |

The inter-assay precision of measuring fluticasone 17β carboxylic acid in stripped and random urine samples was measured and found to be acceptable (Table 12).

TABLE 12

| Replicate # | Level I | Level II | Level III | Random Urine | Level IV |
|---|---|---|---|---|---|
| 1 | 10.1 | 98.0 | 456.0 | | 5080 |
| 2 | 9.9 | 88.0 | 481.0 | 188 | 5040 |
| 3 | 11.3 | 102.0 | 522.0 | 163 | 5070 |
| 4 | 10.2 | 85.5 | 474.0 | 180 | 5550 |
| 5 | 11.9 | 89.5 | 440.0 | 159 | 5480 |
| 6 | 12.4 | 99.9 | 488.0 | 159 | 5520 |
| 7 | 11.1 | 102.0 | 482.0 | 162 | 4660 |
| 8 | 12.9 | 101.0 | 430.0 | 154 | 4860 |
| 9 | 9.4 | 97.8 | 502.0 | 173 | 5300 |
| 10 | 10.5 | 107.0 | 635.0 | 192 | 6050 |
| 11 | 11.1 | 101.0 | 503.0 | 147 | 4550 |
| 12 | 11.4 | 100.0 | 562.0 | 172 | 5380 |
| 13 | 13.8 | 99.3 | 446.0 | 144 | 5290 |
| 14 | 11.1 | 94.3 | 420.0 | 135 | 4400 |
| 15 | 10.0 | 102.0 | 527.0 | 152 | 4250 |
| 16 | 11.9 | 113.0 | 539.0 | 213 | 5470 |
| 17 | 9.3 | 115.0 | 575.0 | 138 | 5200 |
| 18 | 10.1 | 101.0 | 563.0 | 192 | 5480 |
| 19 | 11.5 | 92.2 | 445.0 | 181 | 4760 |
| 20 | 11.4 | 97.6 | 527.0 | 165 | 4940 |
| 21 | | | | 162 | |
| Mean | 11.1 | 99.3 | 500.9 | 166.6 | 5116.5 |
| SD | 1.2 | 7.3 | 55.9 | 20.0 | 445.9 |
| % CV | 10.6% | 7.4% | 11.2% | 12.0% | 8.7% |

The recovery of fluticasone 17β carboxylic acid was measured and found to be acceptable (Table 13).

TABLE 13

| | | Spike | Measured | % Re- |
|---|---|---|---|---|
| Specimen | | (conc) | (conc) | covery |
| High Cntrl + | Pool A neat | Neat (high | | |
| Pool A Urine | | cntrl) | | |
| | 25H:75A | 125.75 | 117 | 93.0% |
| | 50H:50A | 251.5 | 226 | 89.9% |
| | 75H:25A | 377.25 | 333 | 88.3% |
| | High cntrl | 503 | 503 | 100.0% |
| | neat | | | |
| Medium Cntrl + | Pool A neat | Neat | | |
| Pool A Urine | | | | |
| | 25M:75A | 25.25 | 23.3 | 92.3% |
| | 50M:50A | 50.5 | 48.3 | 95.6% |
| | 75M:25A | 75.75 | 71.5 | 94.4% |
| | Med cntrl | 101 | 101 | 100.0% |
| | neat | | | |
| PoolB Urine spiked | Pool B neat | Pool B Neat | | |
| with 250 pg/mL | | | | |
| Diluted with | 25B:75 | 57.5 | 63.9 | 111.1% |
| stripped urine | | | | |
| | 50B:50 | 115 | 110 | 95.7% |
| | 75B:25 | 172.5 | 193 | 111.9% |
| | Pool B spiked | 230 | 230 | 100.0% |
| PoolB Urine spiked | Pool B neat | Neat | | |
| Diluted with | 25B:75 | 135.25 | 138 | 102.0% |
| stripped urine | | | | |
| with 500 pg/mL | | | | |
| | 50B:50 | 270.5 | 259 | 95.7% |
| | 75B:25 | 405.75 | 348 | 85.8% |
| | Pool B spiked | 541 | 541 | 100.0% |
| PoolB Urine spiked | Pool B neat | Pool B Neat | | |
| Diluted with | 25B:75 | 239.25 | 243 | 101.6% |
| stripped urine | | | | |
| with 1000 pg/mL | | | | |
| | 50B:50 | 478.5 | 454 | 94.9% |
| | 75B:25 | 717.75 | 797 | 111.0% |
| | Pool B spiked | 957 | 957 | 100.0% |
| Urine pool B | | Neat | | |
| | | 250 | 230 | 92.0% |
| | | 500 | 541 | 108.2% |
| | | 1000 | 957 | 95.7% |
| | | 5000 | 4510 | 90.2% |

97.9%

The potential interference with testosterone (Table 14) or estrogens (Table 15) was examined and found to be minimal. Interferences were tested by adding blank or high standards of the indicated compound into pooled urine spiked with 50 pg/mL fluticasone-17-beta-carboxylic acid as opposed to 100 pg/mL fluticasone-17-beta-carboxylic acid.

TABLE 14

| URINE-NO PRESERV | | | % Difference | |
|---|---|---|---|---|
| Spec. | F17bCOOH | F17bCOOH + | from F17bCOOH | % |
| ID | Alone | Testosterone | alone | Recovery |
| 1 | 52 | 51.3 | −4.1% | 98.7 |
| 2 | 50.4 | 49.2 | −8.0% | 97.6 |
| 3 | 58.1 | 48.3 | −9.7% | 83.1 |
| Mean | 53.5 | | −7.3% | 93.1 |

TABLE 15

| URINE-NO PRESERV | | F17bCOOH + | % Difference | |
|---|---|---|---|---|
| Spec. | F17bCOOH | Estrogens standard | from F17bCOOH | % |
| ID | Alone | (Estrone & Estradiol) | alone | Recovery |
| 1 | 52 | 51.5 | −3.7% | 99.0 |
| 2 | 50.4 | 57.4 | 7.3% | 113.9 |
| 3 | 58.1 | 58.2 | 8.8% | 100.2 |
| Mean | 53.5 | | 4.1% | 104.4 |

The potential interference with a CAH21 standard (Table 16) or aldosterone (Table 17) was examined and found to be minimal.

TABLE 16

| URINE-NO PRESERV | | F17bCOOH + CAH21 standard | % Difference | |
|---|---|---|---|---|
| Spec. | F17bCOOH | (Androstenedione, 17OH- | from F17bCOOH | % |
| ID | Alone | Progesterone and Cortisol) | alone | Recovery |
| 1 | 52 | 50.2 | −6.2% | 96.5 |
| 2 | 50.4 | 50.4 | −5.8% | 100.0 |
| 3 | 58.1 | 52 | −2.8% | 89.5 |
| Mean | 53.5 | | −4.9% | 95.3 |

TABLE 17

| URINE-NO PRESERV | | | % Difference | |
|---|---|---|---|---|
| Spec. | F17bCOOH | F17bCOOH + | from F17bCOOH | % |
| ID | Alone | Aldosterone | alone | Recovery |
| 1 | 52 | 56.4 | 5.4% | 108.5 |
| 2 | 50.4 | 57.5 | 7.5% | 114.1 |
| 3 | 58.1 | 54.5 | 1.9% | 93.8 |
| Mean | 53.5 | | 4.9% | 105.5 |

The potential interference with DHEA (Table 18), a DOC standard (Table 19), or synthetic glucocorticoids (Table 20) was examined and found to be minimal.

TABLE 18

| URINE-NO PRESERV | | | % Difference | |
|---|---|---|---|---|
| Spec. | F17bCOOH | F17bCOOH + | from F17bCOOH | % |
| ID | Alone | DHEA | alone | Recovery |
| 1 | 52 | 53.9 | 0.7% | 103.7 |
| 2 | 50.4 | 50.1 | −6.4% | 99.4 |
| 3 | 58.1 | 57.3 | 7.1% | 98.6 |
| Mean | 53.5 | | 0.5% | 100.6 |

TABLE 19

| | | F17bCOOH + DOC standard | | |
|---|---|---|---|---|
| URINE-NO PRESERV | | (11-deoxycortisol, | % Difference | |
| Spec. | F17bCOOH | 21-deoxycortisol | from F17bCOOH | % |
| ID | Alone | and corticosterone) | alone | Recovery |
| 1 | 52 | 52.4 | −2.1% | 100.8 |
| 2 | 50.4 | 46.8 | −12.5% | 92.9 |
| 3 | 58.1 | 47.2 | −11.8% | 81.2 |
| Mean | 53.5 | | −8.8% | 91.6 |

TABLE 20

| URINE-NO PRESERV | | F17bCOOH + Synthetic | % Difference | |
|---|---|---|---|---|
| Spec. | F17bCOOH | Glucocorticoids | from F17bCOOH | % |
| ID | Alone | (PROC: 016258) | alone | Recovery |
| 1 | 52 | 62 | 15.9% | 119.2 |
| 2 | 50.4 | 59.9 | 12.0% | 118.8 |
| 3 | 58.1 | 52.7 | −1.5% | 90.7 |
| Mean | 53.5 | | 8.8% | 109.6 |

The linearity was assessed and found to be acceptable (Table 21).

TABLE 21

| | | Measured (Y) | Expected (X) | Percent |
|---|---|---|---|---|
| Specimen | Dilution | (conc) | (conc) | expected |
| Pool 1 | neat | 1840 | 1840 | |
| Spiked Urine | 1:2 | 884 | 920 | 96.1% |
| | 1:4 | 485 | 460 | 105.4% |
| | 1:8 | 234 | 230 | 101.7% |
| | 1:16 | 110 | 115 | 95.7% |
| Pool 2 | neat | 571 | 571 | |
| Urine 1 | 1:2 | 287 | 286 | 100.5% |
| 154 pg/mL | 1:4 | 152 | 143 | 106.4% |
| | 1:8 | 79.4 | 71 | 111.2% |
| | 1:16 | 37 | 36 | 103.6% |
| Pool 3 | neat | 513 | 513 | |
| Urine 2 | 1:2 | 276 | 257 | 107.6% |
| 189 pg/mL | 1:4 | 148 | 128 | 115.4% |
| | 1:8 | 66 | 64 | 103.0% |
| | 1:16 | 33.1 | 32 | 103.1% |
| Pool 4 | neat | 306 | 306 | |
| Urine 3 | 1:2 | 146 | 153 | 95.4% |
| 306 pg/mL | 1:4 | 72.5 | 77 | 94.8% |
| | 1:8 | 40 | 38 | 104.4% |
| | 1:16 | 22.6 | 19 | 118.3% |
| Pool 5 | neat | 524 | 524 | |
| Urine 4 | 1:2 | 279 | 262 | 106.5% |
| 153 pg/mL | 1:4 | 140 | 131 | 106.9% |
| | 1:8 | 63.4 | 66 | 96.8% |
| | 1:16 | 32.7 | 33 | 99.7% |
| Pool 1 | neat | 4580 | 4580 | |
| 5000 pg/mL | 1:2 | 2800 | 2780 | 100.7% |
| | 1:4 | 1210 | 1145 | 105.7% |
| | 1:8 | 589 | 573 | 102.9% |
| | 1:16 | 289 | 286 | 100.9% |
| | neat | 9510 | 9510 | |
| 10000 pg/mL | 1:2 | 5210 | 4755 | 109.6% |
| | 1:4 | 2570 | 2378 | 108.1% |
| | 1:8 | 1120 | 1189 | 94.2% |
| | 1:16 | 543 | 594 | 91.4% |

| | |
|---|---|
| Mean | 103.1% |

The limit of quantitation was assessed and found to be 10.4 pg/mL (Table 22).

TABLE 22

| Replicate # | Minimum (Conc) |
|---|---|
| 1 | 11.7 |
| 2 | 8.9 |
| 3 | 8.6 |
| 4 | 10.8 |
| 5 | 7.7 |
| 6 | 8.3 |
| 7 | 10.6 |
| 8 | 15.3 |
| 9 | 11.9 |
| 10 | 9.5 |
| 11 | 9.8 |
| 12 | 8.3 |
| 13 | 10.6 |
| 14 | 12.8 |
| 15 | 11.1 |
| 16 | 8.2 |
| 17 | 12.1 |
| 18 | 11.3 |
| 19 | 10.4 |
| 20 | 10.1 |
| Mean | 10.4 |
| SD | 1.9 |
| % CV | 17.9% |

The limit of detection was assessed and found to be 8.3 pg/mL, and the critical limit was assessed and found to be 3.7 pg/mL.

Carryover (table 23) was analysed using an estimated carryover influence (ECI) of 5% via the equation:

ECI%=(Relative Carryover)*(Concentration Ratio)*100

TABLE 23

| Repetition # | 1 | 2 | 3 | 4 | 5 | 6 | 7 | 8 | 9 | 10 |
|---|---|---|---|---|---|---|---|---|---|---|
| Concentration | 18200 | 16300 | 19300 | 19100 | 17600 | 19100 | 18400 | 18400 | 18300 | 20000 |
| Peak Area | 6.3E+06 | 5.7E+06 | 6.8E+06 | 6.6E+06 | 6.2E+06 | 4.8E+06 | 4.4E+06 | 4.4E+06 | 4.3E+06 | 4.3E+06 |
| Blank Area | 2.1E+03 | 2.2E+03 | 2.7E+03 | 1.9E+03 | 2.0E+03 | 2.0E+03 | 1.2E+03 | 1.7E+03 | 1.0E+03 | 1.2E+03 |
| Rel Carry (%) | 0.03% | 0.04% | 0.04% | 0.03% | 0.03% | 0.04% | 0.03% | 0.04% | 0.02% | 0.03% |

5% = .033% * Concentration Ratio * 100
Concentration Ratio = 151.5%

Thus, a carryover ratio of 151.5% was supported.

In summary, non-preserved urine can be used effectively. Samples acquired with boric acid preservative indicated an individual bias of −0.6 to 5.0% and a mean bias of 1.4% from non-preserved urine specimen. Samples acquired in glacial acetic acid preservative indicated an individual bias of −18.9 to 17.1% and a mean bias of −1.2% from non-preserved urine specimen. Samples acquired in HCL preservative indicated an individual bias of −14.5 to 9.1% and a mean bias of −5.8% from non-preserved urine specimen. Samples acquired in sodium bicarbonate preservative indicated an individual bias of −94.7 to −92.5% and a mean bias of −93.8% from non-preserved urine specimen. Samples acquired in toluene preservative indicated an individual bias of −10.6 to 9.8% and a mean bias of 0.3% from non-preserved urine specimen. Thus, urine containing boric acid, glacial acetic acid, HCL, or toluene are acceptable. Urine containing sodium bicarbonate preservative is not as acceptable.

Accuracy/Recovery—Since there is no comparative method available, accuracy was assessed by recovery studies. The recovery for six pools ranged from 85.8 to 111.9% with a mean of 97.9%. Acceptance criteria was achieved.

Precision—Intra-assay: n=20. The within run precision ranged from 3.6 to 9.3% for four different pools. Acceptance criteria was achieved.

Precision—Inter-assay: n=20. The between run precision ranged from 7.4 to 12.0% for five different pools. Acceptance criteria was achieved.

Linearity—The linearity for seven pools ranged from 91.4 to 118.3% with a mean of 103.1%. Acceptance criteria was achieved.

Limit of quantitation—The lowest concentration that can be measured with a CV<20% was about 10 pg/mL.

Limit of detection/Critical limit—The critical value and limit of detection was about 3.7 and about 8.3 pg/mL, respectively.

Interferences—The recovery of fluticasone-17-beta-carboxylic acid in samples spiked with testosterone ranged from 83.1-98.7% with a mean of 93.1%. Acceptance criteria was achieved.

Interferences—The recovery of fluticasone-17-beta-carboxylic acid in samples spiked with estrone and estradiol ranged from 99.0-113.9% with a mean of 104.4%. Acceptance criteria was achieved.

Interferences—The recovery of fluticasone-17-beta-carboxylic acid in samples spiked with androstenedione, 170H-progesterone, and cortisol ranged from 89.5-100.0% with a mean of 95.3%. Acceptance criteria was achieved.

Interferences—The recovery of fluticasone-17-beta-carboxylic acid in samples spiked with aldosterone ranged from 93.8-114.1% with a mean of 105.5%. Acceptance criteria was achieved.

Interferences—The recovery of fluticasone-17-beta-carboxylic acid in samples spiked with DHEA ranged from 98.6-103.7% with a mean of 100.6%. Acceptance criteria was achieved.

Interferences—The recovery of fluticasone-17-beta-carboxylic acid in samples spiked with 11-deoxycortisol, 21-deoxycortisol and corticosterone ranged from 81.2-100.8% with a mean of 91.6%. Acceptance criteria was achieved.

Interferences—The recovery of fluticasone-17-beta-carboxylic acid in samples spiked with synthetic corticosteroid standards (PROC: 016258) ranged from 90.7-119.2% with a mean of 109.6%. Acceptance criteria was achieved.

Carryover—Carryover from 10 pools containing 20 ng/mL fluticasone-17-beta-carboxylic acid ranged from 0.02 to 0.04% with a mean of 0.03%. The concentration ratio supported is 151.2. Acceptance criteria was achieved.

### Example 5

### Materials and Methods

### Materials

Fluticasone-17β carboxylic acid (17βFP) was obtained from Synfine Research (Ontario, Canada), and fluorometholone was purchased from Sigma Aldrich (St. Louis, Mo.). Methylene chloride, methanol, and acetonitrile were HPLC grade and obtained from EM Science (Gibbstown, N.J.). All other chemicals were reagent grade. A stock solution of 20 μg/mL 17βFP was prepared in methanol. A 100 ng/mL working solution of 17βFP was prepared by diluting the stock solution 1:200 in 50:50 methanol/H₂O. A 20 μg/mL stock of fluorometholone was prepared in 50:50 methanol/H₂O. A 40 ng/mL working solution of fluorometholone was made by diluting the fluorometholone stock 1:500 in 70:30 methanol/RO water+0.1% formic acid. Charcoal stripped-urine was purchased from SeraCare (Milford, Mass.).

### Sample Preparation

An eight-point calibration curve (0, 10, 25, 100, 200, 1000, 2000, and 10000 pg/mL) and three controls (10, 500 and 5000 pg/mL) were prepared in charcoal stripped-urine and included with each assay run. Aliquots of urine were centrifuged for 5 minutes at 1000g to remove particulate matter. A 1.0 mL fraction of each calibrator, control, and urine sample was transferred into an appropriately labeled 12×75 mm glass tube. Internal standard (100 μL of 40 ng/mL fluorometholone working solution per sample) was added to each calibrator, control, and urine sample followed by gentle vortexing and incubation for 5 minutes at ambient temperature. Acetonitrile with 0.1% HCl (1.5 mL/sample) was then added, and tubes were vortexed and centrifuged at 1500g for 10 minutes. Supernatants were transferred into clean 13×100 mm glass tubes, and 4 mL methylene chloride was added to each tube. The samples were vortex-mixed and centrifuged at 1000g for 5 minutes. The upper aqueous layer was aspirated and discarded. The methylene chloride fractions were washed sequentially with 1.0 mL of 1 N HCL, and 1.0 mL of H₂O wherein the aqueous layer was aspirated and discarded. The washed methylene chloride fractions were dried under nitrogen at 45° C., and the dried extract was reconstituted in 100 μL of 70:30 methanol/H₂O with 0.1% formic acid. The reconstituted extracts were gently vortexed, centrifuged at 1000g for 5 minutes, and transferred to autosampler plates.

### LC-MS/MS

All LC-MS/MS experiments were performed with a CTC-PAL autosampler for sample introduction, Perkin-Elmer series 200 pumps, and an ABI 4000™ Q-trap tandem mass spectrometer (Applied Biosystems) operating with electrospray ionization and positive-mode multiple reaction monitoring. Sixty μL reconstituted extract was injected. Fluticasone-17β-carboxylic acid and fluorometholone were chromatographically resolved from other sample components using a reversed-phase analytical column (3 μm Pursuit XRs C18; 50×2.0 mm ID; Varian, Inc.) combined with a precolumn filter (C18; 4×2 mm ID; Phenomenex®) and gradient elution with mobile phase buffer A (10:90 acetonitrile/H₂O with 0.1% formic acid) and buffer B (90:10 acetonitrile/H₂O with 0.1% formic acid). The mobile phase was delivered at a flow rate of 250 μL/minute with the following conditions: 20% buffer B for 0.3 minutes, step gradient to 50% buffer B for 2.2 minutes, step gradient to 83% buffer B for 2.5 minutes, final reequilibration with 20% buffer B for 0.5 minutes. Total instrument analysis time, including sample introduction and run time, was 5.5 minutes per sample.

The mass spectrometer operating conditions consisted of a source heater probe of 450° C., Turbolonspray voltage of 4200 V, entrance potential of 10 V, curtain gas setting of 20, gas one setting of 50, gas two setting of 55 and collision gas CAD setting of medium. Data acquisition and quantitative processing were conducted using Analyst™ 1.4.2 software (Applied Biosystems). Multiple-reaction monitoring ion transitions included Q1 m/z ratio of 453.2 and Q3 m/z ratio of 293.1 for 1713FP (declustering potential of 60 V, collision energy of 21 V, collision cell exit potential of 7 V and retention time of 2.5 min), as well as Q1 m/z ratio of 377.0 and Q3 m/z ratio of 279.0 for fluorometholone (declustering potential of 35 V, collision energy of 37 V, collision cell exit potential of 12 V and retention time of 2.2 minutes).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing steroid treatment compliance of a human instructed to self administer a steroid, wherein said method comprises determining whether or not a biological sample from said human contains a detectable level of said steroid or a metabolite of said steroid, wherein the presence of said detectable level indicates that said human is in compliance with the instructed steroid treatment, and wherein the absence of said detectable level indicates that said human is not in compliance with the instructed steroid treatment.

2. The method of claim 1, wherein said steroid is fluticasone propionate.

3. The method of claim 1, wherein said metabolite is fluticasone 170β carboxylic acid.

4. The method of claim 1, wherein said human has asthma.

5. The method of claim 1, wherein said human has an allergic disease.

6. The method of claim 1, wherein said biological sample is a urine sample.

7. A method for assessing the metabolic activity of CYP3A4 in a human who received fluticasone propionate, wherein said method comprises (a) using mass spectrometry to determine the level of said fluticasone propionate and the level of a metabolite of said fluticasone propionate present in a biological sample from said human and (b) diagnosing said human as having poor CYP3A4 metabolic activity if the ratio of said fluticasone propionate to said metabolite is greater than that compared to normal humans and diagnosing said human as having active CYP3A4 metabolic activity if the ratio of said fluticasone propionate to said metabolite is less than that compared to normal humans.

8. The method of claim 7, wherein said method comprises communicating said diagnosis to said human.

9. The method of claim 7, wherein said method comprises inserting a notation of said diagnosis into a medical record for said human.
